(19) United States
(12) Patent Application Publication
(10) Pub. No.: US 2007/0033716 A1
(43) Pub. Date: Feb. 15, 2007

(54) Goggles with removable frame and methods of making and using the same

(75) Inventor: John Dondero, Ketchum, ID (US)

Correspondence Address: John M. Janeway;GRAYBEAL JACKSON HALEY LLP, Suite 350, 155 - 108th Avenue NE, Bellevue, WA, 98004-5901, US

(73) Assignee: Eye Safety Systems, Inc.

(21) Appl. No.: 11/437,227

(22) Filed: May 18, 2006

Related U.S. Application Data
(60) Provisional application No. 60/682,793, filed on May 18, 2005.

(51) Int. Cl. A61F 9/02

(52) U.S. Cl. 2/426

(57) ABSTRACT

A removable goggle frame that may be releasably fastened to a goggle having a frame that retains a lens is provided. The removable frame may be stiffer (harder) than the goggle frame, and, when fastened to the goggle, may protect the goggle from damage cause by undesirable impacts by shielding the goggle frame and/or stiffening the goggle frame. The removable frame may also be more flexible (softer) than the goggle frame, and, when fastened to the goggle, may provide a more comfortable fit for the user. A removable drape that may be releasably fastened to a releasably attached frame is also provided. The drape may be configured to hang below the goggle to protect the user's face when worn, and to be releasably fastened over the lens to protect the lens when the user places the goggle elsewhere, such on his/her helmet as he/she wears the helmet.

### INCORPORATION BY REFERENCE OF RELATED APPLICATIONS

This application claims priority from commonly owned U.S. Provisional Patent Application 60/682,793, filed 18 May 2005, and titled Goggles With Removable Frame And Methods Of Making And Using The Same, presently pending, which is incorporated herein by reference in its entirety and for all its teachings and disclosures.

Each of the following is hereby incorporated by reference in its entirety for all its teachings and disclosures: U.S. Pat. No. 6,490,729 issued on 10 Dec. 2002; U.S. Pat. No. 6,708,340 issued on 23 Mar. 23, 2004; U.S. Publication No. US 2002/0157175 A1 published on 31 Oct. 2002; U.S Publication No. US 2004/0083540 A1 published on 6 May 2004; U.S Publication No.: US20050015862-A1 published on 27 January 2005; U.S Pat. No. 6,048,410 issued on 11 Apr. 2000; and U.S. Pat. No. 6038,707 issued on 21 Mar. 2000.

### BACKGROUND

Many goggles having a soft frame that molds well to the face and is very comfortable for a user. However, these goggles can have problems when used with high impact activities, such as paintball games, practicing military combat exercises, or participating in actual combat. The relatively soft frame can deform during or after impact of a projectile or debris. In addition, the soft frame can sometimes be unable to retain the lens within the frame during or after such impact. John, I chose to not combine these last two sentences because the idea expressed in these two sentences is broader that the idea expressed in one, combined sentence. Broader is good here.

Similarly many goggles are held on a user's head above his eyes or on a helmet when the user is not wearing the goggle to protect his eyes. In such situations, the goggle remains exposed to the environment and can be damaged by debris or other materials in the environment. For example, a person inside a tank having his goggles placed over his helmet and above his eyes can inadvertently scratch or otherwise damage the lens of the goggle by bumping the goggle into a component of the tank.

In view of the above, there is a need for an improved goggle and related methods that remedy these and other problems.

### SUMMARY

One aspect of the invention provides a frame that is releasably attachable to a goggle having a lens and a frame that holds the lens, wherein the releasably attachable frame comprises a body sized and configured to protect the goggle frame from impacts when the releasably attachable frame is fastened to the goggle and a user wears the goggle with the lens positioned in front of an eye. The releasably attachable frame also comprises at least one attachment component sized and configured to engage the goggle to releasably fasten the body to the goggle. The releasably attachable frame is typically stiffer (harder) than the frame of the goggle and can protect the goggle from undesirable impacts by shielding the frame of the goggle from such impacts and by supporting the frame of the goggle during such impacts. Because the frame may be releasably fastened to the goggle, a user can modify, as desired, the degree of protection afforded the goggle by replacing a frame with a different frame having a different stiffness or coverage configuration.

In another aspect of the invention a releasably attachable frame may be fastened to a goggle to form an interface between the goggle and the user when the user wears the goggle with the lens positioned in front of an eye. In this aspect of the invention, the releasably attachable frame comprises a body having a face-contoured component that forms a substantially air-tight seal with the user's face. The releasably attachable frame is typically more flexible (softer) than the frame of the goggle and can provide a more comfortable fit for a user. Because the frame may be releasably fastened to the goggle, a user can modify, as desired, the goggle to change the fit of the goggle in response to a change in the environment or purpose for wearing the goggle, by replacing a frame with a different frame. In addition, a user can modify, as desired, the goggle to comfortably fit another's face by replacing a frame with a different frame having a different flexibility.

In yet another aspect of the invention a drape may be releasably fastened to a releasably attachable frame having a lens and a frame that holds the lens, to protect the goggle or a substantial component of the goggle when the goggle is held on a helmet worn by the user or on other equipment of the user, or placed away from the user, such as a duffel bag or work bench. The drape covers the goggle or just specific components of the goggle, such as the lens and then is releasably fastened to the goggle to keep the drape over the desired components of the goggle.

In another aspect of the invention, a method for making a goggle includes releasably attaching a frame to a goggle having a lens and a frame that holds the lens. The method also includes removing a releasably attachable frame from a goggle and fastening another releasably attachable frame to the goggle.

In yet another aspect of the invention, a method for making a goggle includes releasably attaching a drape to a releasably attachable frame, covering one or more of the goggle's components with the drape, and hanging the drape from the goggle to protect a user's face.

### BRIEF DESCRIPTION OF THE FIGURES

FIG. 1 is an exploded, perspective view of a goggle according to an embodiment of the invention.

FIG. 2 is a perspective view of the goggle in FIG. 1.

FIG. 3 is a perspective view of a goggle according to another embodiment of the invention.

FIG. 4 is a perspective view of a goggle according to yet another embodiment of the invention.

FIG. 5 is a cross-sectional view of the goggle in FIG. 2 taken along the line 5-5, according to an embodiment of the invention.

FIG. 6 is a cross-sectional view of the goggle in FIG. 2 taken along the line 6-6, according to an embodiment of the invention.

FIG. 7 is a perspective view of a releasably attachable frame according to an embodiment of the invention.

FIG. 8 is a perspective view of goggle according to another embodiment of the invention.

### DETAILED DESCRIPTION

Various modifications to the disclosed embodiments will be readily apparent and the generic principles herein may be applied to other embodiments and applications without departing from the spirit and scope of the present discussion. Thus, the present discussion is not intended to be limited to the embodiments shown, but is to be accorded the widest scope consistent with the principles and features disclosed herein.

One aspect of the invention provides a removable goggle frame that may be releasably fastened to a goggle having a frame that retains a lens. In some embodiments, the removable frame is stiffer (harder) than the goggle frame, and, when fastened to the goggle, protects the goggle from damage cause by undesirable impacts by shielding the goggle frame and/or supporting the goggle frame. In other embodiments, the removable frame is more flexible (softer) than the goggle frame, and, when fastened to the goggle, provides a more comfortable fit for the user.

Another aspect of the invention provides a removable drape that may be releasably fastened to a releasably attachable frame. In some embodiments, the drape is configured to hang below the releasably attachable frame to protect the user's face when the releasably attachable frame is fastened to a goggle and the combination is worn, and configured to be releasably fastened over a lens of either the goggle, releasably attachable frame, or both to protect the lens when the user places the releasably attachable frame and goggle elsewhere, such on his/her helmet as he/she wears the helmet. In other embodiments, the drape covers substantially all of the releasably attachable frame and goggle.

In certain embodiments, the present application is directed to a removable frame alone, while in other embodiments, the application is directed to a combination of the removable goggle frame and a goggle that it may be attached to. Likewise, in certain embodiments, the present application is directed to a removable drape alone, while in other embodiments, the application is directed to a combination of the removable drape and a goggle that it may be attached to. The present application is further directed to methods of making the various elements of the systems and apparatus, as well as to methods of using the same.

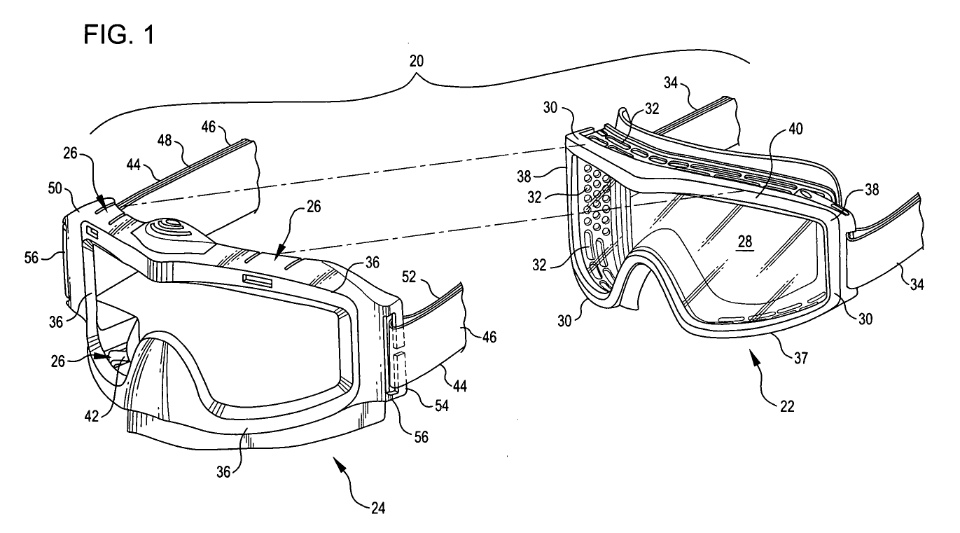

FIG. 1 is an exploded, perspective view of a goggle 20 according to an embodiment of the invention. The goggle 20 includes a goggle 22, and a releasably attachable frame 24 that may be fastened to the goggle 22 to protect the goggle 22 from undesirable impacts. The goggle 22 may be any desired goggle, such as an all-purpose goggle or a goggle designed for a specific purpose, such as a military ballistic goggle. In some embodiments of the goggle 20, an attachment component 26 releasably fastens the frame 24 to the goggle 22.

The goggle 22 includes a lens 28 and a frame 30 that holds the lens 28. In some embodiments, the goggle 22 includes vents 32 to allow air to enter and exit a region between a user's eye, the lens 28, and the frame 30. The goggle 22 may also include a goggle-retention member 34 to hold the goggle 22 to a user when worn by the user. The frame 30 may be sized and configured to retain the lens 28 in front of an eye of a user when the goggle is worn by the user to protect his/her eye. The frame 30, often but not always, is made of material that substantially flexes under moderate pressure to allow the frame 30 to comfortably fit many different faces.

The releasably attachable frame 24 protects the frame 30, and thus the goggle 22, from undesirable impacts by shielding the frame 30 from such impacts and by supporting the frame 30 during such impacts. Examples of such undesirable impacts include a paint ball striking the frame 30 while participating in a paintball game, or debris striking the frame 30 while participating in military combat practice exercises or in actual combat exercises. Because the frame 30 is often substantially flexible, such impacts to the frame 30 can damage the goggle 22 by, for example, separating all or a portion of the frame 30 from the lens 28.

The releasably attachable frame 24 includes a body 36 that may be made of any desirable material having any desired stiffness to protect the frame 30. Stiffness is a measure of a material's ability to resist deforming or flexing when subjected to a force. For example, most paper clips are stiffer than most wood twigs having the same dimensions because more force is required to bend the paper clip than the wood twig. In this and other embodiments of the releasably attachable frame 24, the body 36 is made of any desirable plastic having a stiffness at least 10 times greater than the stiffness of the frame 30. In other embodiments, the plastic material has a stiffness that ranges between slightly less than the stiffness of the frame 30 to less than 10 times the stiffness of the frame 30. In still other embodiments of the releasably attachable frame 24, the body 36 is made of any desirable metal having a stiffness that ranges between slightly less than the stiffness of the frame 30 to more than the stiffness of the frame 30. In still other embodiments of the releasably attachable frame 24, the body 36 may be made from any desirable combination of materials that provides any desired stiffness to protect the frame 30.

The body 36 may be sized and configured as desired to shield the frame 30 from undesirable impacts and to support the frame 30 during such an impact. For example, in this and other embodiments of the frame 24, the body 36 substantially covers the frame 30 (see FIGS. 2, 5 and 6) when the releasably attachable frame 24 is fastened to the goggle 22. In other embodiments of the releasably attachable frame 24, the body 36 covers one or more segments (full sides, such as the top, lateral sides or bottom) of the frame 30. For example, the body 36 may include separate pieces (not shown), each releasably fastened to a respective one of the following segments of the frame 30 to protect these segments: a bottom segment 37 and a brow segment 40. As another example, the body 36 may include a single piece (not shown) releasably fastened to the brow segment 40 and both side segments 38 of the frame 30 to protect these segments of the frame 30.

Still referring to FIG. 1, in this and other embodiments of the goggle 20, the releasably attachable frame 24 includes the attachment component 26. The frame 24 may include any desired number of the attachment components 26, each located as desired. For example in this and other embodiments of the releasably attachable frame 24, the frame 24 includes four detents 42 (only one shown in FIG. 1 for clarity) sized and configured to releasably engage the frame 30 (discussed in greater detail in conjunction with FIG. 5). In other embodiments of the releasably attachable frame 24, the frame 24 includes an attachment component (not shown) sized and configured to releasably engage another substantial component of the goggle 22, such as the lens 28.

In other embodiments of the goggle 20, the goggle 22 includes the attachment component 26, and the goggle 22 includes any desired number of the attachment components 26, each located as desired. In still other embodiments, the frame 24 may be releasably fastened to the goggle 22 using any desired fastening technique capable of retaining the frame 24 to the goggle 22 during and after an undesirable impact.

Because the frame 24 is releasably fastened to the goggle 22, a user can modify, as desired, the degree of protection afforded the goggle 22 by replacing a frame 24 with a different frame 24 having a different stiffness or coverage configuration.

Still referring to FIG. 1, in this and other embodiments of the releasably attachable frame 24, the frame 24 includes a frame-retention member 44 to hold the frame 24 to a user when worn by the user. For example, in some embodiments the frame-retention member 44 is an elastic strap 46 having a length, a first end 48 connected to a right side 50 of the body 36, and a second end 52 connected to a left side 54 of the body 36. In some embodiments of the goggle 20, the frame-retention member 44 is the only component of the goggle 20 that holds the goggle 20 to a user when the goggle is worn by the user. In other embodiments of the frame 24, the right side 50, the left side 54 or both sides 50 and 54 include a receiver 56 sized and configured to receive and hold the goggle-retention member 34. In such embodiments the goggle-retention member 34 may be the only component of the goggle 20 that holds the goggle 20 to a user when the goggle is worn by the user.

Other embodiments of the goggle 20 are possible. For example, the goggle 20 may include clips (not shown) or other elements (not shown) to retain the goggle 20 to other protective gear such as helmets, ear protectors and the like. In addition, the goggle 22 can be sized and configured to fit close to a user's eyes or further away from a user's eyes to accommodate glasses or other eyewear that a user may choose to wear. Also, the releasably attachable frame 24 can have a variety of different colors or other artistic schemes such that a user can switch external goggle frames for reasons related to stylistic choices (as opposed to, or in addition to, more functional reasons such as differences in protective configurations, wear factors, etc.).

FIG. 2 is a perspective view of the goggle 20 in FIG. 1 showing the releasably attachable frame 24 fastened to the goggle 22. In some embodiments, the goggle 20 includes a fan (not shown) to urge air into and out of the region 57 between a user's eye, the lens 28, and the frame 30. This may be desirable when some embodiments of the frame 24 that include a body 36 having a vent cover 58 to protect a vent 32 and region 57, are fastened to the goggle 22. In some embodiments of the frame 24, the vent covers 58 can sufficiently impede the flow of air into and out of the region 57 to prevent adequate ventilation of the region 57. Without adequate ventilation, condensation from perspiration can form on the inside side surface of the lens and interfere with a user's vision. To restore adequate ventilation to the region 57, the fan may be mounted to the goggle 20.

In this and other embodiments of the goggle 20, the fan may be mounted to the frame 30 of the goggle under the grill 60 of the releasably attachable frame 24. The fan may be mounted to the frame 30 using any desired fastening technique. In other embodiments, the fan may be mounted to the releasably attachable frame 24 using any desired fastening technique. In some embodiments, the grill 60 includes vents 62 to allow air to enter or exit the fan.

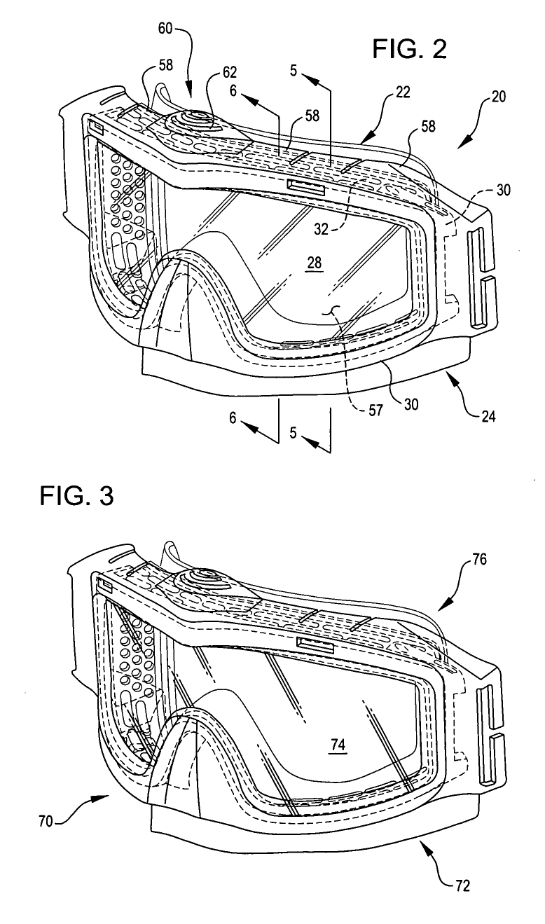

FIG. 3 is a perspective view of a goggle 70 according to another embodiment of the invention. The goggle 70 is similar to the goggle 20 in FIGS. 1 and 2, except the releasably attachable frame 72 includes a lens 74, and the goggle 76 does not. With the lens 74 a part of the releasably attachable frame 72, one can easily change lenses of the goggle 70 as desired, for example, to match changing light conditions.

The lens 74 can protect a user's eyes from bright or harmful light as well as particles suspended in the ambient air and other matter. The lens 74 is typically made of any desired light transmissive material. For example, if desired, the lens 74 can be substantially clear and transmit substantially all light that contacts the lens. However, when specific conditions like bright light, very low light or high intensity collimated light, e.g., laser beams, or objects with high kinetic energy exist, lenses specifically designed for the condition may be used. For example, a tinted or polarized lens may be made from a light tinting or light polarizing material, or made by attaching a tinted or polarized film to the lens 74. Furthermore, the tinting material or film may filter a narrow range of light wavelengths such as those corresponding to the light wavelengths emitted by lasers or welding torches. Also, the material of the lens 74 can be impact resistant as well as shatter proof like ballistic grade lenses. The lens 74 may also selectively attenuate infrared light or light having a wavelength greater than approximately 670 nanometers. In addition, the lens 74 may attenuate ultraviolet light or light having a wavelength less than approximately 420 nanometers. Or, the lens 74 may attenuate both infrared and ultraviolet light. In addition, the lens 74 can include “tear aways” (not shown)—removable sheets covering the outside surface of the lens. By simply pulling off a “tear away”, a muddied or oiled lens can be quickly and easily cleaned.

The lens 28 in FIGS. 1 and 2 of the goggle 22 in FIGS. 1 and 2 may be similar to the lens 74. Therefore, the discussion about the lens 74 also applies to the lens 28.

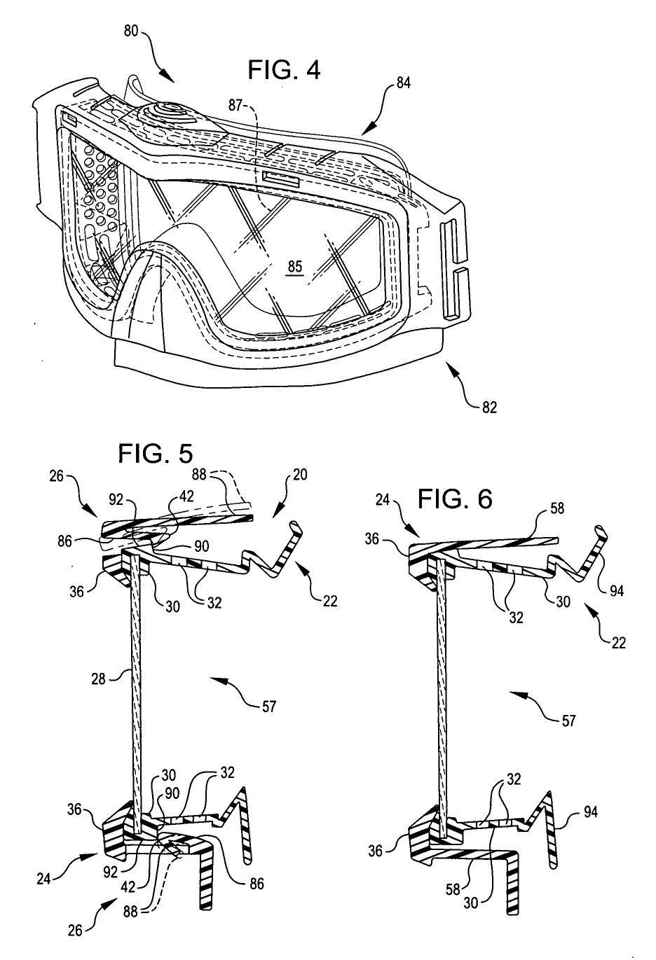

FIG. 4 is a perspective view of a goggle 80 according to yet another embodiment of the invention. The goggle 80 is similar to the goggle 20 in FIGS. 1 and 2, and goggle 70 in FIG. 3, except both the releasably attachable frame 82 and the goggle 84 each includes a lens 85 and 87, respectively. With both lenses 85 and 87, the goggle 80 can provide more protection from undesirable impacts than the goggles 20 and 70, and the lenses 85 and 87 each can provide protection from certain types of light that the other lens 87 and 85 does not and thus when combined can provide protection from a broader range of light types.

FIG. 5 is a cross-sectional view of the goggle 20 in FIG. 2 taken along the line 5-5, according to an embodiment of the invention. In this and other embodiments of the releasably attachable frame 24, the attachment component 26 (two shown) releasably engages the frame 30 of the goggle 22 to fasten the frame 24 to the goggle 22. Furthermore, in this and other embodiments of the attachment component 26, each component 26 is sized and configured to hold the frame 30 to the frame 24 without requiring that the frame 30 have a corresponding structure specifically designed to receive the attachment component 26.

For example, in this and other embodiments, each attachment component 26 includes a detent 42, a cantilever 86, and a release tab 88. Each detent 42 is sized and configured to hold the frame 30 of the goggle 22 against the body 36 of the releasably attachable frame 24 when the detent 42 is positioned to releasably fasten the frame 24 to the goggle 22. Each cantilever 86 has an end from which a respective detent 42 extends, and is sized and configured to position the respective detent 42 to contact the frame 30 when the detent 42 holds the frame 30 against the body 36. Each release tab 88 is sized and configured to move a respective detent 42 relative to the frame 30 to release the detent's hold on the frame 30. As shown in FIG. 5, when each attachment component 26 fastens the body 36 to the frame 30, each detent 42 contacts a back surface 90 of the frame 30. And as shown in FIG. 5 in phantom, each detent 42 is moved away from the frame 30 to separate the detent 42 from the back surface 90 and thus release the detent's hold on the frame 30.

Because most conventional goggles have a frame that includes a back surface similar to the surface 90 of the goggle 22, the attachment component 26 may be used to releasably fasten the frame 24 to different goggles 22 as desired. In other embodiments of the goggle 20, the goggle 22 may include a receiver specifically designed to receive and hold a detent or other structure of the releasably attachable frame 24.

In this and other embodiments, a user can fasten a desired releasably attachable frame 24 to a goggle 22 that does not have a frame 24 already fastened to it by aligning the frame 24 with the goggle 22 and then moving the frame 24 toward the goggle 22. As the body 36 closes in toward the frame 30, each detent contacts an outer surface 92 of the frame 30, which causes a respective cantilever 86 to flex away from the frame 30. Then, when the body 36 contacts the frame 30, each cantilever 86 urges a respective detent 42 into a position where the detent 42 contacts the back surface 90. To release the detent's hold on the frame 30, the release tab 88 is moved away from the frame 30, which causes a respective detent 42 to separate from the back surface 90.

FIG. 6 is a cross-sectional view of the goggle in FIG. 2 taken along the line 6-6, according to an embodiment of the invention. In this and other embodiments, the body 36 includes a vent cover 58 to protect the region 57 and a vent 32 of the goggle 22 from paint, projectiles, undesirable debris or other undesired elements.

The vent cover 58 may be sized and configured to extend any desired length over the vent 32 and to be located any desired distance away from the vent 32. For example in this and other embodiments, the vent cover 58 extends from the body 36, covers the vent 32, and is located less than an inch from the vent 32. With the vent cover 58 covering the vent 32, substantial protection is afforded the frame 30, vent 32 and region 57. In this configuration, however, airflow through the vent 32 can be substantially impeded, causing inadequate ventilation of the region 57. Without adequate ventilation, condensation from perspiration can form on the inside surface of the lens and interfere with a user's vision. To reduce the restriction on the airflow, other embodiments may include a vent cover that extends over a portion of the vent 32, or that is located an inch or more away from the vent 32, or that is geometrically configured to urge airflow through the vent, or any combination of these.

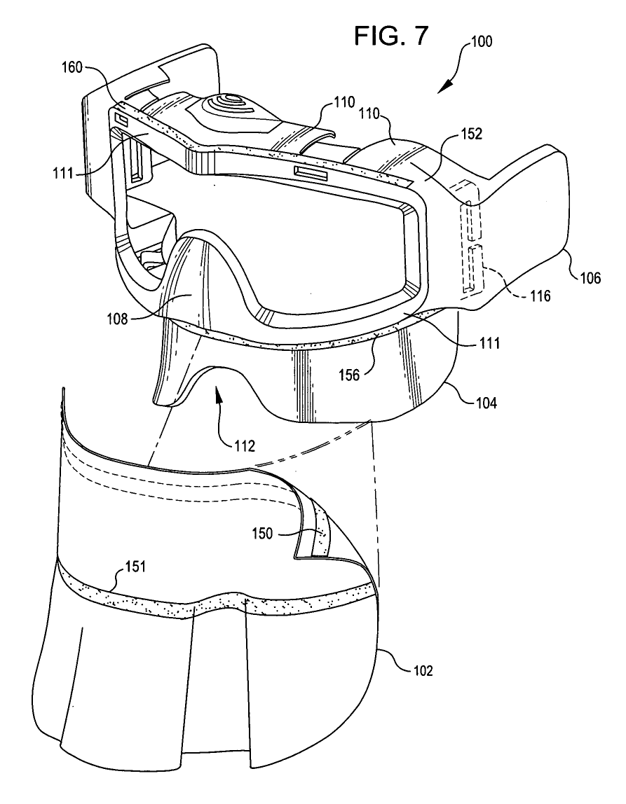

In other embodiments of the releasably attachable frame 24, such as the embodiment shown in FIG. 7, the vent cover 58 may cover the vent 32 and a portion of a face-contoured component 94 of goggle 22.

FIG. 7 is a perspective view of a releasably attachable frame 100 according to an embodiment of the invention. In this and other embodiments, the releasably attachable frame 100 is similar to the frame 24 in FIGS. 1, 2, 5 and 6, except the frame 100 includes the following components, a drape 102, cheek guard 104, goggle-retention guard 106 and nose guard 108, as well as a vent cover 110 that extends to cover a portion of a face-contoured component (not shown in FIG. 7) as discussed in conjunction with FIG. 6. In other embodiments, the releasably attachable frame 100 may include one or more of these components in any desired combination.

In this and other embodiments of the frame 100, the body 111 includes a cheek guard 104 to protect a user's cheek (not shown) when the releasably attachable frame 100 is fastened to a goggle (not shown) and the goggle is worn by a user (not shown). The cheek guard 104 may be sized and configured as desired to provide any desired degree of protection. For example, in this and other embodiments the check guard 104 extends over a portion of a user's cheek, and includes an opening 112 configured to align with a user's mouth. In other embodiments, the cheek guard 104 may not include the opening 112 for a user's mouth and may extend over a user's mouth to cover a portion or all of the mouth, or the cheek guard 104 may further extend to cover a user's cheek and lower jaw.

In this and other embodiments, the releasably attachable frame 100 includes a drape 102 to protect a user's face (not shown) and other desired body parts, and, when the releasably attachable frame 100 is fastened to the goggle 22 (shown in FIG. 1 but not in FIG. 7), to protect the goggle 22 or components of the goggle 22 such as the lens 28 (shown in FIG. 1 but not in FIG. 7). When the releasably attachable frame 100 is fastened to the goggle 22 and the goggle 22 is worn to protect an eye of the user, the drape 102 may hang from the releasably attachable frame 100 and extend over the user's face and or other desired bodily parts, such as the neck. When the releasably attachable frame 100 is fastened to the goggle 22 and the combination is held elsewhere on the user such as a helmet, or held or placed away from the user, such as a duffel bag or work bench, the drape 102 may be releasably fastened over the lens 28 or other goggle components.

The drape 102 may be made of any desirable material. For example in this and other embodiments, the drape may be made of a protective cloth such as ballistic cloth. In other embodiments, the drape 102 may be made of any soft, compliant material such as a thermoplastic rubber or a thermoplastic elastomer.

The drape 102 may be sized and configured as desired to provide any desired degree of protection. For example, in this and other embodiments the drape 102 is large enough to extend over the portion of a user's face not covered by the goggle 22 and releasably attachable frame 100, and the user's neck. In still other embodiments, the drape 102 may be sized and configured to just cover the lens 28 when the drape 102 is fastened over the goggle 22. In such a configuration, the drape 102 may or may not cover the user's face when the drape hangs from the releasably attachable frame 100. In still other embodiments, the drape 102 may extend over only a portion of a user's face as desired when the drape 102 hangs from the releasably attachable frame 100.

The drape 102 includes a drape-attachment component, and may be releasably fastened to the releasably attachable frame 100 at any desired location. For example, in this and other embodiments, the drape 102 includes a first drape-attachment component 150 that may be releasably fastened to the cheek guard 104, and a spaced apart second drape-attachment component 151 that may be releasably fastened to a top portion 152 of the body 111. The first drape-attachment component 150 may be Velcro that fastens to a corresponding Velcro strip 156 of the releasably attachable frame 100 when the drape 102 hangs from the frame 100 to protect a user, and when the drape 102 extends over the lens 28. The second drape-attachment component 151 may be Velcro that fastens to a corresponding Velcro strip 160 of the releasably attachable frame 100 when the drape 102 extends over the lens 28. In other embodiments, the Velcro strip 160 may be located adjacent the Velcro strip 156 and the second drape-attachment component 151 may be fastened to the strip 160 after the drape 102 has been wrapped around the goggle 22. In still other embodiments the drape may be releasably attached to the releasably attachable frame 100 with snaps.

In other embodiments of the releasably attachable frame 100, the drape 102 may be releasably fastened to the body 111 using any desired fastening techniques. In still other embodiments, the drape 102 may be fastened to the frame 100 in a more permanent manner, such as adhesive or stitches.

Still referring to FIG. 7, in this and other embodiments of the frame 100, the body 111 includes a goggle-retention guard 106 to protect the connection of the frame 100 to a frame-retention member (not shown) or a goggle-retention member (not shown), or to protect the connection of a goggle frame (not shown) to a goggle-retention member. The guard 106 may be sized and configured as desired to provide any desired degree of protection. For example, in this and other embodiments the guard 106 extends over the receiver 116 of the frame 100, and over the location where the goggle frame is connected to the goggle-retention member when the goggle (not shown) and frame 100 fastened together. In other embodiments, the guard 106 may extend over a portion of a user's ear (not shown), or to cover a user's ear.

In this and other embodiments of the frame 100, the body 111 includes a nose guard 108 to protect a user's nose (not shown) when the releasably attachable frame 100 is fastened to a goggle and the goggle is worn by a user. The nose guard 108 may be sized and configured as desired to provide any desired degree of protection. For example, in this and other embodiments the nose guard 108 covers a user's nose, and includes an opening (not shown) to allow the user to breath through the nose while wearing the frame 100. In other embodiments, the nose guard 108 may not include the opening. In still other embodiments the nose guard 108 may extend over a portion of the user's nose.

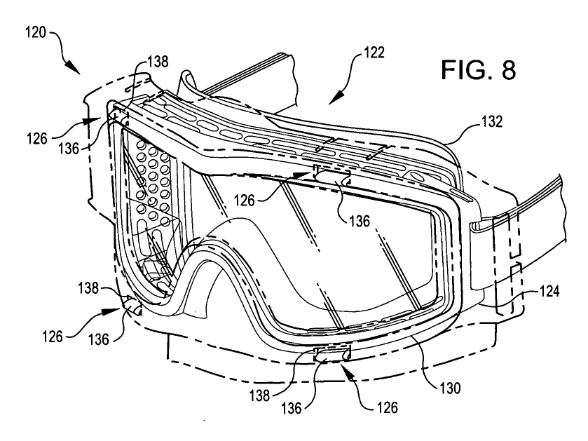

FIG. 8 is a perspective view of a goggle 120 according to another embodiment of the invention. The goggle 120 includes a goggle 122, and a releasably attachable frame 124 that may be fastened to the goggle 122 to form a substantially air-tight seal with a user's face (not shown) when a user wears the goggle 120. The goggle 120 is similar to the goggle 70 in FIG. 3 except the releasably attachable frame 124 forms the internal portion of the goggle 120 that contacts the user's face when the user wears the goggle 120, and the goggle 122 forms the external portion of the goggle 120 that protects the releasably attachable frame 124 from undesirable impacts. In this and other embodiments of the goggle 20, an attachment component 126 releasably fastens the frame 124 to the goggle 122.

The goggle 122 includes a lens 128 that is similar to the lens 74 in FIG. 3, and may be any desired goggle, such as an all-purpose goggle or a goggle designed for a specific purpose, such as a military ballistic goggle.

The releasably attachable frame 124 includes a body 130 having a face-contoured component 132. The body 130 may be made of any desirable material having any desired flexibility to comfortably support the goggle 122 when a user wears the goggle 120, and the face-contoured component 132 may be made of any desirable material having any desired pliability to comfortably form a substantially air-tight seal with the face of different users. Flexibility and pliability are a measure of a material's ability to deform when subjected to a force. Compared to each other, a pliable material is very flexible, and a flexible material may not be pliable. For example, a rubber band is typically pliable because it substantially deforms when subjected to a small amount of force, and a plastic ruler is typically flexible but not pliable because the ruler will start to deform with a moderate amount of force. In this and other embodiments of the releasably attachable frame 124, the body 130 is made of any desirable rubber or plastic that is more flexible than the goggle 122, and the face-contoured component 132 is made of any desirable rubber or plastic that is pliable or more flexible that the body 130.

The face-contoured component 132 may be sized and configured as desired. For example, in this and other embodiments of the releasably attachable frame 124, the face-contoured component 132 forms a perimeter around both eyes of a user when the goggle 120 is worn with the lens 128 positioned in front of both eyes. Thus, the face-contoured component 124 can form a substantially air-tight seal around the users eyes to prevent paint, projectiles, undesirable debris or other undesired elements from entering the region between a user's eye, the lens 128, and the frame 124. In other embodiments of the releasably attachable frame 124, the face-contoured component 132 contacts one or more segments (not shown) of a user's face to form a substantially air-tight seal with the segment. For example, the component 132 may extend only across the top of the body 130 and contact the brow (not shown) of a user to form a substantially air-tight seal with the brow. As another example, the component 132 may include two separate pieces, one that extends across the top of the body 130 and contacts the brow of a user, and another one that extends across the bottom of the body 130 and contacts the nose of a user. This may be desirable to provide adequate ventilation to the region between a user's eye, the lens 128, and the frame 124.

Still referring to FIG. 1, in this and other embodiments of the goggle 120, the releasably attachable frame 124 includes the attachment component 126. The frame 124 may include any desired number of the attachment components 126, each located as desired. For example in this and other embodiments of the releasably attachable frame 124, the frame 124 includes four detents 136 sized and configured to releasably engage the goggle 122. The detents 136 and their engagement with the goggle 122 are similar to the detents 42 in FIG. 5 and their engagement with the frame 30 in FIG. 5, except that detents 136 extend through slots 138 in the goggle 122 when the detents 136 hold the goggle 122 to the releasably attachable frame 124. In other embodiments of the releasably attachable frame 124, the frame 124 includes an attachment component (not shown) sized and configured to releasably engage another substantial component of the goggle 22, such as the lens 128.

In other embodiments of the goggle 120, the goggle 122 includes the attachment component 126, and the goggle 122 includes any desired number of the attachment components 126, each located as desired. In still other embodiments, the frame 124 may be releasably fastened to the goggle 122 using any desired fastening technique capable of retaining the frame 124 to the goggle 122.

Because the frame 124 is releasably fastened to the goggle 122, a user can modify, as desired, the goggle 120 to change the fit of the goggle in response to changes in the environment, or purpose for wearing the goggle 120 by replacing a frame 124 with a different frame 124 having a different body 130 and/or configuration for the face-contoured component 132. In addition, a user can modify, as desired, the goggle 120 to comfortably fit another's face by replacing a frame 124 with a different frame 124 having a different flexibility and or pliability in the face-contoured component 132.

Still referring to FIG. 8, In this and other embodiments of the goggle 120, the releasably attachable frame 124 may also include vents to allow air to enter and exit a region between a user's eye, the lens 128, and the frame 124, and that are similar to the vents 32 in FIGS. 1-6 and discussed elsewhere herein. In other embodiments of the goggle 120, the releasably attachable frame 124 may not include any vents.

From the foregoing, it will be appreciated that, although specific embodiments have been discussed herein for purposes of illustration, various modifications may be made without deviating from the spirit and scope of the discussion herein. Accordingly, the systems and methods include such modifications as well as all permutations and combinations of the subject matter set forth herein and are not limited except as by the appended claims.

What is claimed is:

1. A releasably attachable frame that is releasably attachable to a goggle having a lens and a goggle frame that holds the lens, the releasably attachable frame comprising:
a body sized and configured to protect the goggle frame from impacts when the releasably attachable frame is fastened to the goggle and a user wears the goggle with the lens positioned in front of an eye; and
an attachment component sized and configured to engage the goggle to releasably fasten the body to the goggle.

2. The releasably attachable frame of claim 1 wherein the body is stiffer than the goggle frame.

3. The releasably attachable frame of claim 1 wherein the body is sized and configured to support the goggle frame to protect it from impacts when the releasably attachable frame is fastened to the goggle.

4. The releasably attachable frame of claim 1 wherein the body is sized and configured to shield the goggle frame to protect it from impacts when the releasably attachable frame is fastened to the goggle.

5. The releasably attachable frame of claim 1 wherein the body is sized and configured to substantially cover a segment of the goggle frame when the releasably attachable frame is fastened to the goggle.

6. The releasably attachable frame of claim 1 wherein the body is sized and configured to substantially cover substantially all of the frame that is exposed to the outside environment when the releasably attachable frame is not fastened to the goggle frame.

7. The releasably attachable frame of claim 1 wherein the attachment component engages the goggle frame when the releasably attachable frame is fastened to the goggle.

8. The releasably attachable frame of claim 1 wherein the attachment component comprises a detent sized and configured to contact the goggle frame to releasably fasten the body to the goggle.

9. The releasably attachable frame of claim 1 wherein the attachment component comprises a detent sized and configured to hold the goggle frame against the body to releasably fasten the body to the goggle.

10. The releasably attachable frame of claim 1 wherein the attachment component comprises:
a detent sized and configured to hold the goggle frame against the body to releasably fasten the body to the goggle, and
a flexible cantilever having an end that the detent extends from, the cantilever being sized and configured to position the detent to hold the goggle frame.

11. The releasably attachable frame of claim 1 wherein the attachment component comprises:
a detent sized and configured to hold the goggle frame against the body to releasably fasten the body to the goggle,
a flexible cantilever having an end that the detent extends from, the cantilever being sized and configured to position the detent to hold the goggle frame, and
a release tab sized and configured for a user to move to release the detent's hold on goggle frame.

12. The releasably attachable frame of claim 1 wherein the attachment component comprises an elastic strap having a length, a first end connected to a left side of the releasably attachable frame, and a second end connected to a right side of the releasably attachable frame.

13. The releasably attachable frame of claim 1 wherein the body comprises a vent cover sized and configured to extend over a vent of the goggle when the releasably attachable frame is fastened to the goggle.

14. The releasably attachable frame of claim 13 wherein the vent cover is sized and configured to substantially cover the goggle's vent when the releasably attachable frame is fastened to the goggle.

15. The releasably attachable frame of claim 14 wherein the vent cover is sized and configured to allow air to flow through the goggle's vent when the releasably attachable frame is fastened to the goggle.

16. The releasably attachable frame of claim 1 wherein the body comprises a nose guard sized and configured to extend over a user's nose when the releasably attachable frame is fastened to the goggle and the user wears the goggle with the lens positioned in front of an eye.

17. The releasably attachable frame of claim 16 wherein the nose guard is sized and configured to cover the user's nose when the releasably attachable frame is fastened to the goggle and the user wears the goggle with the lens positioned in front of an eye.

18. The releasably attachable frame of claim 1 wherein the body comprises a cheek guard sized and configured to extend over a user's cheek when the releasably attachable frame is fastened to the goggle and the user wears the goggle with the lens positioned in front of an eye.

19. The releasably attachable frame of claim 18 wherein the cheek guard is sized and configured to cover the user's cheek when the releasably attachable frame is fastened to the goggle and the user wears the goggle with the lens positioned in front of an eye.

20. The releasably attachable frame of claim 18 wherein the cheek guard is sized and configured to extend over both cheeks of a user when the releasably attachable frame is fastened to the goggle and the user wears the goggle with the lens positioned in front of an eye.

21. The releasably attachable frame of claim 1 wherein the body comprises a mount sized and configured to accommodate a fan to the releasably attachable frame, wherein the fan is sized and configured to urge air through a vent of the goggle.

22. The releasably attachable frame of claim 1 wherein the body comprises a goggle-retention guard sized and configured to extend over a location of the goggle where a goggle-retention member of the goggle connects to the goggle frame, when the releasably attachable frame is fastened to the goggle.

23. The releasably attachable frame of claim 1 wherein the body comprises a receiver sized and configured to receive a goggle-retention member of the goggle to allow the goggle-retention member to hold the releasably attachable frame to a user when the releasably attachable frame is fastened to the goggle.

24. The releasably attachable frame of claim 1 further comprising a frame-retention member comprising an elastic strap having a length, a first end connected to a left side of the releasably attachable frame, and a second end connected to a right side of the releasably attachable frame.

25. The releasably attachable frame of claim 1 further comprising a drape fastened to the body and sized and configured to extend over a user's face when the releasably attachable frame is fastened to the goggle and the user wears the goggle with the lens positioned in front of an eye.

26. The releasably attachable frame of claim 25 wherein the drape is sized and configured to cover the user's face and extend over a user's neck when the releasably attachable frame is fastened to the goggle and the user wears the goggle with the lens positioned in front of an eye.

27. The releasably attachable frame of claim 25 wherein the drape is releasably fastened to the body.

28. The releasably attachable frame of claim 27 wherein the drape comprises:
a first drape-attachment component sized and configured to fasten the drape to the releasably attachable frame when the drape hangs from the releasably attachable frame and extends over a user's face, and when the drape extends over a component of the goggle; and
a second drape-attachment component sized and configured to fasten the drape to the releasably attachable frame when the drape extends over a component of the goggle.

29. The drape of claim 28 wherein the drape covers the lens when the second drape-attachment component fastens the drape to the releasably attachable frame.

30. The drape of claim 28 wherein the first and second drape-attachment components comprise Velcro.

31. The releasably attachable frame of claim 1 further comprising a lens held by the body.

32. A releasably attachable frame that is releasably attachable to a goggle having a lens and a goggle frame that holds the lens, the releasably attachable frame comprising:
a body having a face-contoured component that forms a substantially air-tight seal with a user's face when the frame is fastened to the goggle and a user wears the goggle with the lens positioned in front of an eye; and
an attachment component configured to engage the goggle to releasably fasten the body to the goggle.

33. The releasably attachable frame of claim 32 wherein the body is substantially flexible.

34. The releasably attachable frame of claim 32 wherein the body is substantially more flexible than the goggle frame.

35. The releasably attachable frame of claim 32 wherein the face-contoured component is pliable.

36. The releasably attachable frame of claim 32 wherein the face-contoured component forms a perimeter.

37. The releasably attachable frame of claim 32 wherein the attachment component engages the goggle frame when the releasably attachable frame is fastened to the goggle.

38. The releasably attachable frame of claim 32 wherein the attachment component comprises a detent sized and configured to contact the goggle frame to releasably fasten the body to the goggle.

39. The releasably attachable frame of claim 32 wherein the attachment component comprises a detent sized and configured to hold the goggle frame against the body to releasably fasten the body to the goggle.

40. The releasably attachable frame of claim 32 wherein the body comprises a vent sized and configured allow air to enter and exit a region between a user's eye, the goggle's lens and the releasably attachable frame when the releasably attachable frame is fastened to the goggle.

41. The releasably attachable frame of claim 32 further comprising a frame-retention member sized and configured to retain the releasably attachable frame to a user.

42. The releasably attachable frame of claim 41 wherein the frame-retention member comprises an elastic strap having a length, a first end connected to a left side of the releasably attachable frame, and a second end connected to a right side of the releasably attachable frame.

43. The releasably attachable frame of claim 32 further comprising a lens held by the body.

44. A goggle comprising:
a first goggle frame;
a lens;
a second releasably attachable frame releasably attachable to the first goggle frame, the second releasably attachable frame comprising:
a body sized and configured to protect the first goggle frame from impacts when the second releasably attachable frame is fastened to the first goggle frame and a user wears the goggle with the lens positioned in front of an eye; and
an attachment component sized and configured to engage the first goggle frame to releasably fasten the body to the first goggle frame.

45. The goggle of claim 44 wherein the first goggle frame holds the lens.

46. The goggle of claim 44 wherein the second releasably attachable frame holds the lens.

47. The goggle of claim 44 further comprising a second lens, wherein the second releasably attachable frame holds the second lens and the first goggle frame holds the other lens.

48. The goggle of claim 44 wherein the first goggle frame comprises a vent sized and configured to allow air to enter and exit a region between a user's eye, the goggle's lens and the first goggle frame.

49. The goggle of claim 44 further comprising a fan mounted to the first goggle frame, and sized and configured to urge air through the vent of the goggle.

50. The goggle of claim 44 further comprising a frame-retention member connected to the first goggle frame, and sized and configured to retain the first goggle frame to a user.

51. The goggle of claim 44 further comprising a frame-retention member connected to the second releasably attachable frame, and sized and configured to retain the second releasably attachable frame to a user.

52. The goggle of claim 50 wherein the frame-retention member comprises an elastic strap having a length, a first end connected to a left side of the first goggle frame, and a second end connected to a right side of the first goggle frame.

53. The goggle of claim 51 wherein the frame-retention member comprises an elastic strap having a length, a first end connected to a left side of the second releasably attachable frame, and a second end connected to a right side of the second releasably attachable frame.

54. A goggle comprising:
a first goggle frame;
a lens;
a second releasably attachable frame releasably attachable to the first goggle frame, the second releasably attachable frame comprising:
a body having a face-contoured component that forms a substantially air-tight seal with a user's face when the frame is fastened to the goggle and a user wears the goggle with the lens positioned in front of an eye; and
an attachment component sized and configured to engage the first goggle frame to releasably fasten the body to the goggle.

55. The goggle of claim 54 wherein the first goggle frame holds the lens.

56. The goggle of claim 54 wherein the second releasably attachable frame holds the lens.

57. The goggle of claim 54 further comprising a second lens, wherein the second releasably attachable frame holds the second lens and the first goggle frame holds the other lens.

58. The goggle of claim 54 wherein the second releasably attachable frame comprises a vent sized and configured to allow air to enter and exit a region between a user's eye, the goggle's lens and the second releasably attachable frame.

59. A method comprising:
attaching a releasably attachable frame to a goggle having a lens and a goggle frame to hold the lens, wherein the releasably attachable frame comprises a body sized and configured to protect the goggle frame from impacts when the frame is fastened to the goggle and a user wears the goggle with the lens positioned in front of an eye.

60. The method of claim 59 further comprising removing the releasably attachable frame from the goggle.

61. The method of claim 59 further comprising:
removing the releasably attachable frame from the goggle, and
attaching another releasably attachable frame to the goggle, wherein the releasably attachable frame comprises a body sized and configured to protect the goggle frame from impacts when the frame is fastened to the goggle and a user wears the goggle with the lens positioned in front of an eye.

62. The method of claim 59 further comprising positioning the goggle and the releasably attachable frame such that the lens of the goggle is positioned in front of a user's eye and the goggle contacts the users face.

63. The method of claim 59 further comprising mounting the goggle to a user's face.

64. The method of claim 63 wherein mounting the goggle to a user's face occurs before attaching the releasably attached frame to the goggle.

65. The method of claim 63 wherein attaching the releasably attachable frame to the goggle occurs before mounting the goggle to a user's face.

66. A method comprising:
attaching a releasably attachable frame to a goggle having a lens and a goggle frame to hold the lens, wherein the releasably attachable frame comprises a body having a face-contoured component that forms a substantially air-tight seal with a user's face when the releasably attachable frame is fastened to the goggle and a user wears the goggle with the lens positioned in front of an eye.

67. The method of claim 66 further comprising removing the releasably attachable frame from the goggle.

68. The method of claim 66 further comprising:
removing the releasably attachable frame from the goggle, and
attaching another second releasably attachable frame to the goggle, wherein the second releasably attachable frame comprises a body having a face-contoured component that forms a substantially air-tight seal with a user's face when the second releasably attachable frame is fastened to the goggle and a user wears the goggle with the lens positioned in front of an eye.

69. The method of claim 66 further comprising positioning the goggle and the releasably attachable frame such that the lens of the goggle is positioned in front of a user's eye and the goggle contacts the users face.

70. The method of claim 66 further comprising mounting the goggle to a user's face.

71. The method of claim 70 wherein mounting the goggle to a user's face occurs before attaching the releasably attached frame to the goggle.

72. The method of claim 70 wherein attaching the releasably attachable frame to the goggle occurs before mounting the goggle to a user's face.
